(19) United States
(12) Patent Application Publication
(10) Pub. No.: US 2009/0204144 A1
(43) Pub. Date: Aug. 13, 2009

(54) DEVICE FOR THE CONTROL OF URINARY INCONTINENCE

(76) Inventor: Juan Carlos DE FRANCESCO, Buenos Aires (AR)

Correspondence Address: AMIN Talati, LLC, 444 NORTH ORLEANS STREET, SUITE 400, CHICAGO, IL, 60654, US

(21) Appl. No.: 12/366,485

(22) Filed: Feb. 5, 2009

(30) Foreign Application Priority Data
Feb. 7, 2008 (AR) MUT20080100541
Jan. 12, 2009 (MX) MX/A/2009/002066

(51) Int. Cl. A61B 17/122

(52) U.S. Cl. 606/201

(57) ABSTRACT

A device for the control of urinary incontinence includes a support (1) having a transverse opening (3) positionable around the body (10a) of a penis (10) so that an area of the penis (10) adjacent to the urethra ductus (10b) is subject to the action of a mobile arm (5). The mobile arm (5) includes a compressing head (6) which has a prominent sector (6a) for compressing said urethra ductus (10b) and exerting adjustable pressure to control urinary incontinence. The compressing head (6) can further include relief sectors (6b) which prevent the compression of the cavernous sectors (10a) of the penis (10) thereby allowing their normal blood circulation. In addition, the displacement of the body (10a) of the penis (10) against the area (2a) of the inner rim (2) opposite the mobile arm (5) is comfortable thanks to the presence of a wall or soft surface (4).

### FIELD OF THE INVENTION

The present device is related to the field of daily life and medical sciences and hygiene needs and within said field, to the external means that allow controlling urinary incontinence.

More particularly, it consists of a device for the control of urinary incontinence, which allows for the compression of the urethra ductus and the regulation of the intensity of the pressure exerted.

### BACKGROUND OF THE INVENTION

1. Prior Art

As it is well known, there are millions of people who suffer from urinary incontinence, which seriously affects their life quality. This is the case of people who have been treated for different disorders of the urinary tract—such as prostate or bladder cancer surgery—and who, in spite of having overcome the disease, are still affected by incontinence problems.

Some ways of coping with the problem are based on the use of diapers, which result in the discomfort and limitations they generate.

Up to now, the devices known are disclosed by the U.S. Pat. Nos. 2,455,859 and 2,533,924 based on compressing means with external nozzles connections.

U.S. Pat. No. 2,756,753 shows a device with a belting or tape and a little nipper.

U.S. Pat. No. 3,147,754 shows a set of claws with a set of runner toothing.

U.S. Pat. No. 3,203,421 shows a set of articulated jaws with a side thread exerting uneven and one-sided compression.

U.S. Pat. No. 3,926,175 shows a compressing means with magnetic control.

U.S. Pat. No. 4,534,353 shows a device, which lacks threading, therefore, its regulation capacity is scarce.

U.S. Pat. Nos. D299,168, 4,942,886 and 4,800,879 show jaws with one end articulated and the other end toothed. U.S. Pat. No. 6,349,727 shows a device with elastic jaws and an end with levers.

Finally, the device in U.S. Pat. No. 4,880,016 is based on a tape with a set of openings and an anchorage.

2. Advantages and Purposes

An object of the present device is to allow adjustable control of urinary incontinence.

An advantage of the present invention is the high capability to adjust the pressure it exerts on the urethra ductus. This is due to the action of a compressing head driven by a mobile arm, which positional variation is based on a thread.

Another advantage is that the inclusion of prominent sectors and relief sectors in the compressing head, allows compression of the area of the urethra ductus only, keeping the penis cavernous sectors free from pressure.

Another advantage is that the incorporation of a soft wall or surface allows preventing lesions or discomfort of the penis while pressure is exerted on its body.

Another advantage is that the compressing head may be kept fixed while it turns round the mobile arm that drives it. This prevents attrition between the above mentioned head and the body of the penis.

Another advantage is that it lacks springs, toothing or any other element that might cause lesions to the user. Except the command of the mobile arm, there are no other projecting parts that may cause discomfort.

A further advantage is that its size is extremely reduced.

Other additional advantages are its constitutive simplicity and the low production cost.

### BRIEF DESCRIPTION OF THE DRAWINGS

FIG. 1 comprises drawings A and B, where:

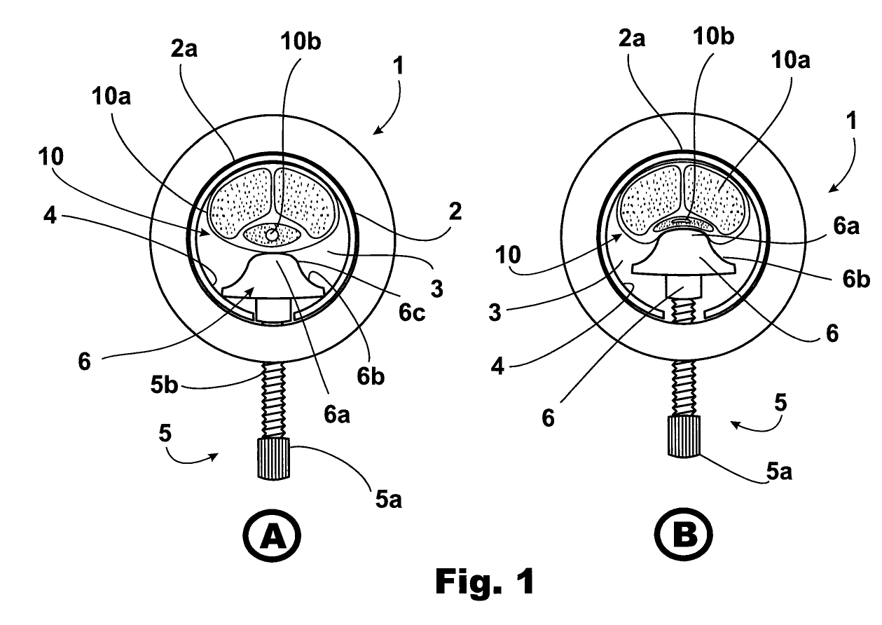

Drawing A is a front elevation view of the present device with a penis—represented in a transverse section—arranged into the transverse opening or support passage; and

Drawing B is another front elevation view of the present device wherein it can be clearly appreciated how the prominent sector of the compressing head acts by closing the urethra ductus, while the relief sectors prevent the total release of the cavernous sectors of said penis.

FIG. 2 comprises drawings A and B, where:

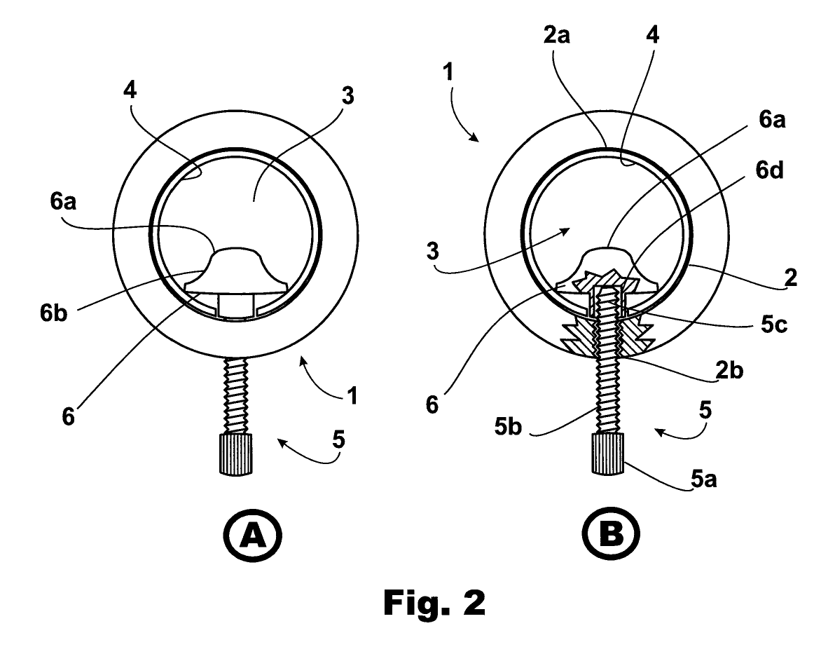

Drawing A is a front elevation view of the present device with the mobile arm completely projected out of the transverse opening leaving it free to place the body of the penis; and

Drawing B is a partial longitudinal section, which allows appreciating the relation between the support, the mobile arm and the compressing head.

FIG. 3 comprises drawings A, B and C, where:

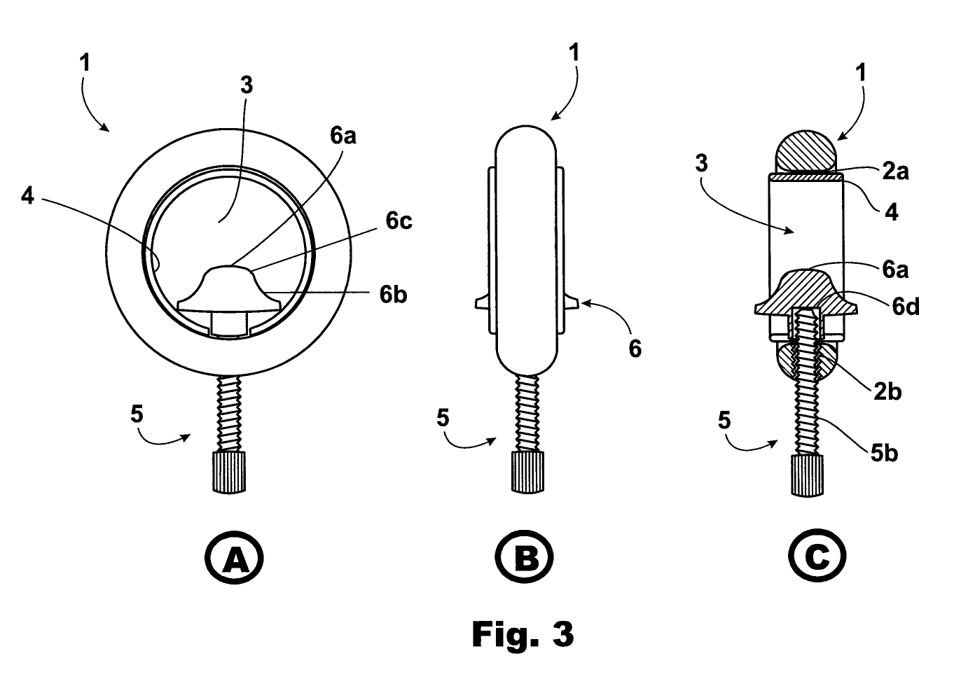

Drawing A is a front elevation view as in drawing 1A;

Drawing B is a side elevated view of the present device; and

Drawing C is a transverse section, which allows appreciating the device structure in detail.

FIG. 4 is an exploded view of the present device in a side elevation view.

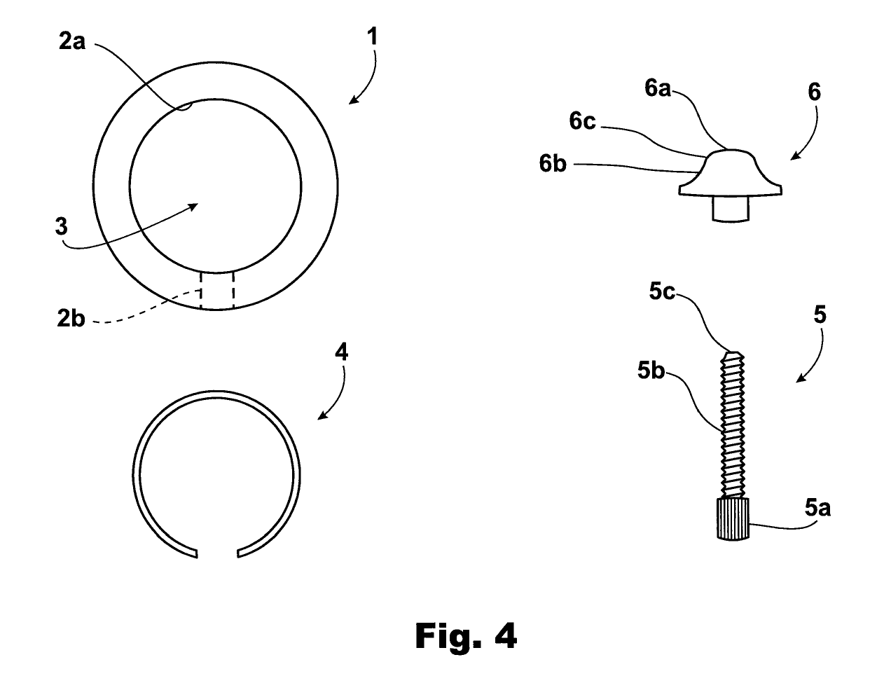

FIG. 5 comprises drawings A and B, where:

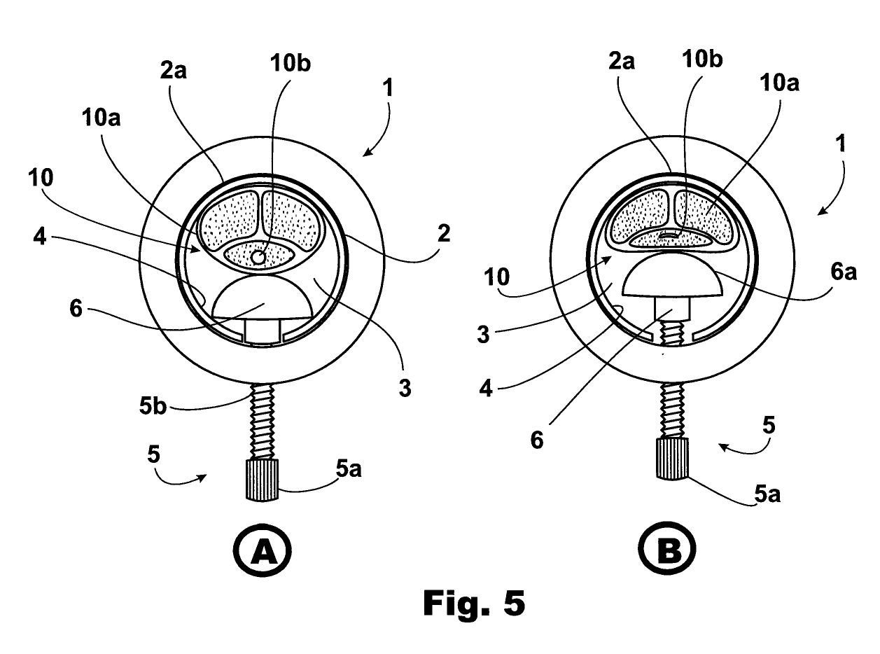

Drawing A is a front elevation view of another embodiment of the present device with a penis—represented in a transverse section—arranged into the transverse opening or support passage; and

Drawing B is another front elevation view of the device of drawing A in which it can be clearly appreciated how the compressing head acts by closing the urethra ductus.

FIG. 6 comprises drawings A and B, where:

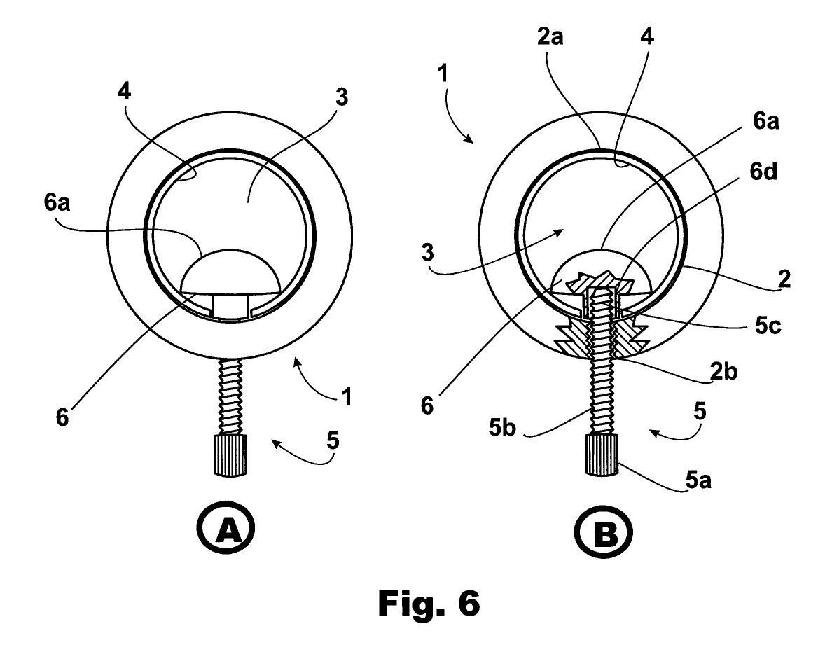

Drawing A is a front elevation view of the device in FIG. 5 with the mobile arm totally projected outside the transverse opening leaving it free to place the body of the penis; and

Drawing B is a partial longitudinal section, which allows appreciating the relation between the support, the mobile arm and the compressing head.

FIG. 7 comprises drawings A, B and C, where:

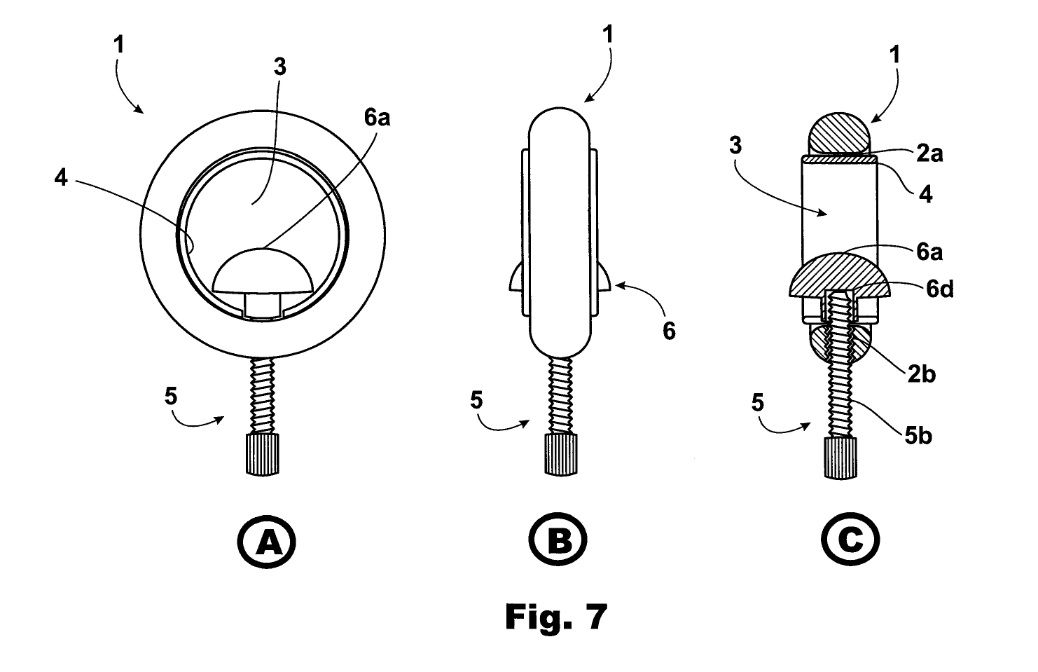

Drawing A is a front elevation view as in drawing 5A;

Drawing B is a side elevation view of the device in FIG. 5; and

Drawing C is a transverse section, which allows appreciating the structure of the device in detail

FIG. 8 is an exploded view of the device in FIG. 5 in a side elevation view.

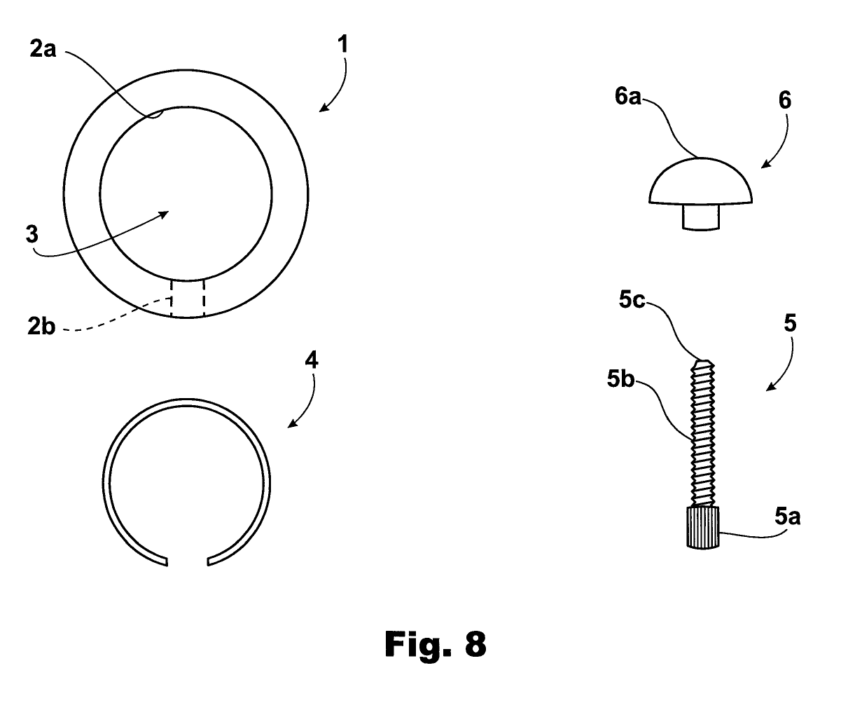

In the different figures, the same numbers and and/or reference letters indicate equal or corresponding parts.

### REFERENCE NUMERALS IN DRAWINGS

(1) Support.

(2) Inner rim of the support (1).
(2a) Area opposite to the compressing head (6).
(2b) Fixed thread.

(3) Transverse opening or passage for the body (10a) of the penis (10).

(4) Soft wall.

(5) Compression mobile arm.
(5a) Command of the mobile arm (5).
(5b) Mobile thread.
(5c) Mobile end.

(6) Compressing head.
(6a) Prominent sector of the compressing head (6).
(6b) Relief sector.
(6c) Transition rim.
(6d) Free turn connection.

(10) Application penis.
(10a) Cavernous sectors (10).
(10b) Urethra ductus.

### DESCRIPTION

In general terms, the present invention consists of a device for control of urinary incontinence that comprises a support (1) in which transverse opening (3) is arranged the body (10a) of a penis (10) so that, its area adjacent to the urethra ductus (10b), is subject to the action of a mobile arm (5) which compressing head (6) has a prominent sector (6a) able to compress said urethra ductus (10b), exerting an adjustable pressure for the control of urinary incontinency. Detailed description:

More particularly, the present device is applicable to the body of the penis (10) so that the closure of the urethra ductus (10b) is obtained.

This device comprises a support (1) that, even though it may have different shapes and configurations, in the embodiment described, forms a body mainly annular—as a fence—, which outer surface is substantially free of edges or rims susceptible to causing lesions on the user's body.

The above mentioned support (1) has some inner rims (2) which delimit a transverse opening (3) or passage able to admit the body of a penis (10) through it and receive a transverse section of the penis, so that an area adjacent to the urethra ductus (10b) is exposed to the action of the means (5) and (6) of the present device.

In said transverse opening (3) or passage, a mobile arm (5) operates having positional variation means (5b) which allow its alternative advance or reverse projection on the above mentioned opening (3).

These positional variation means (5b) are given by a mobile thread (5b) which relates to a fixed thread (2b) provided by the support (1). For the displacement of said mobile arm (5), there is a command (5a) that may consist of a clamp, throttle, faceted body, lever, knob, etc.

The mobile arm (5) ends in a mobile end (5c) where a compressing head (6) is mounted, so that said compressing head (6) may have free turn capacity relative to said mobile end (5c). It has also been considered an embodiment in which the compressing head (6) has a certain capacity of axial displacement in relation to the mobile arm (5) to facilitate its adequate position.

In addition, the compressing head (6) has a surface partially or totally convex (6a) which provides higher efficiency to its compressing action on the area adjacent to the urethra ductus (10b).

In a preferred embodiment, the compressing head (6) comprises a prominent sector (6a)—applicable to the urethra ductus (10b)—in which adjacence there are relief sectors (6b) which prevent the compression of the cavernous bodies (10a). The transition between both sectors (6a) and (6b) is provided by some transition rims (6c) which lack edges.

It has been provided that, at least, the area opposite (2a) to the mobile arm (5) is soft so that the penis (10) can be compressed without suffering lesions.

Therefore, the inner rim (2) of the support (1) may have a soft wall (4), cover or surface that covers it totally or partially.

### Operation:

The transverse opening (3) or passage allows that through the opening (3) the application penis (10) may be placed, until being arranged in such a way that a sector adjacent to the urethra ductus (10b) is subject to the action of the control means (5) and (6).

In these conditions, by means of the respective command (5a), the mobile arm is driven (5) so that its mobile thread (5b) moves through the fixed thread (2b) of the support (1). This allows the advance of the compressing head (6) until its wall or convex surface (6a) contacts the area adjacent to the urethra (10b), from there onwards, being able to adjust the pressure exerted by the above mentioned head (6).

Once the contact is made, the compressing head (6) may stop turning although the mobile arm (5) continues turning due to the free turn connection (6d), which allows preventing any attrition or friction with the body (10a) of the penis (10).

In the case of the compressing head (6) with prominent (6a) and relief (6b) sectors, this prominent sector (6a) is the one that compresses the area of the urethra (10b), whereas the relief sectors (6b) prevent the compression of the cavernous sectors (10a) of the penis (10), allowing their normal blood circulation.

In addition, the movement of the body (10a) of the penis (10) against the opposite area (2a) of the inner rim (2) is comfortable, thanks to the presence of a soft wall (4) or surface.

It has also been provided that the outer surface of the support (1) is free of edges or rims that might affect areas adjacent to the user's body.

Finally, it has been provided the possibility that the device has a set of two or more interchangeable compressing heads (6) with different shapes and for different uses, anatomies, times, etc.

Undoubtedly, upon putting the present invention into practice, modifications may be introduced regarding certain construction details and form, without leaving the essential principles that are clearly explained in the claims below.

What is claimed is:

1. A device for the control of urinary incontinence, applicable around the body of a penis and having the capacity to compress selectively an area adjacent to the urethra ductus, comprising:
a support having a transverse opening able to admit the body of the penis there through; and
a mobile arm for applying adjustable compression to at least a portion of the area adjacent to the urethra ductus of the penis, said mobile arm including a command means, a positional variation means, and a compressing head,
wherein, in use, the mobile arm is positioned adjacent to at least a portion of the area adjacent to the urethra ductus of the penis, and
wherein at least a portion of an inner area of the support opposite the mobile arm is soft.

2. The device for the control of urinary incontinence, according to claim 1, wherein the support comprises a fence that delimits a passage able to receive the penis there through, said fence having a compressing mobile arm which, through said passage, acts on an area of the penis where the urethra ductus is arranged.

3. The device for the control of urinary incontinence, according to claim 1, wherein the support comprises an annular fence.

4. The device for the control of urinary incontinence, according to claim 1, wherein the transverse opening is a passage able to allow the entry of a penis through it, until the area adjacent to the urethra of said penis is exposed to the action of the compressing mobile arm.

5. The device for the control of urinary incontinence, according to claim 1, wherein the positional variation means of the compressing mobile arm are provided by a thread.

6. The device for the control of urinary incontinence, according to claim 1, wherein the compressing head of the mobile arm is at least partially convex.

7. The device for the control of urinary incontinence, according to claim 1, wherein the compressing head of the mobile arm is prominent in a sector that compresses the urethra ductus of the penis.

8. The device for the control of urinary incontinence, according to claim 1, wherein the compressing head of the mobile arm comprises:
a prominent sector able to compress the urethra ductus of the penis; and
adjacent relief sectors which prevent the compression of cavernous bodies of said penis.

9. The device for the control of urinary incontinence, according to claim 7, wherein the compressing head of the mobile arm is centrally prominent.

10. The device for the control of urinary incontinence, according to claim 1, wherein the compressing head has free turn capacity with respect to the mobile arm at which end it is mounted.

11. The device for the control of urinary incontinence, according to claim 1, wherein the compressing head has a certain capacity of axial displacement with respect to the mobile arm.

12. The device for the control of urinary incontinence, according to claim 1, wherein the command means comprises a command clamp.

13. The device for the control of urinary incontinence, according to claim 1, wherein the command means comprises a throttle type clamp.

14. The device for the control of urinary incontinence, according to claim 1, wherein the command means comprises a faceted clamp.

15. The device for the control of urinary incontinence, according to claim 1, wherein the command means comprises an arm.

16. The device for the control of urinary incontinence, according to claim 1, wherein an inner area opposite the mobile arm is soft.

17. The device for the control of urinary incontinence, according to claim 1, wherein a whole inner part of the support is soft.

18. The device for the control of urinary incontinence, according to claim 1, wherein in the support, at least, an inner area opposite the mobile arm comprises a soft cover.

19. The device for the control of urinary incontinence, according to claim 1, wherein a body of the support forms an outer surface substantially free of edges.

20. The device for the control of urinary incontinence, according to claim 1, wherein the device comprises a set of interchangeable compressing heads.
